(19) United States
(12) Patent Application Publication
(10) Pub. No.: US 2026/0235878 A1
(43) Pub. Date: Aug. 13, 2026

(54) EYEGLASSES AND METHOD FOR THE SAME

(71) Applicant: Sony Group Corporation, Tokyo (JP)

(72) Inventors: Dunai Fuentes HITOS, Zurich (CH); Lev MARKHASIN, Stuttgart (DE); Justinas MISEIKIS, Zurich (CH); Diederik Paul MOEYS, Zurich (CH); Stefan UHLICH, Stuttgart (DE)

(21) Appl. No.: 19/156,665

(22) PCT Filed: Feb. 29, 2024
(86) PCT No.: PCT/EP2024/055279

§ 371 Date: Aug. 14, 2025

(30) Foreign Application Priority Data
Mar. 1, 2023 (EP) 23159417.7

(51) Int. Cl. G02B 27/01; G02B 27/00; G06F 1/16; G06F 3/01; G06F 3/04817; G16H 20/30

(52) U.S. Cl. CPC G02B 27/0172; G02B 27/0093; G06F 1/163; G06F 3/011; G06F 3/04817; G16H 20/30; G02B 2027/0138; G02B 2027/014; G02B 2027/0141; G02B 2027/0178

(57) ABSTRACT

Eyeglasses are provided. The eyeglasses include a display and a camera configured to capture an environment of the eyeglasses. Additionally, the eyeglasses include processing circuitry coupled to the camera and the display. The processing circuitry is configured to determine, based on image data of the camera, whether the environment offers a user of the eyeglasses an opportunity to perform a physical health exercise. If it is determined that the environment offers an opportunity to perform a physical health exercise, the processing circuitry is configured to control the display to output one or more graphical elements related to the opportunity to perform a physical health exercise.

### FIELD

The present disclosure relates to eyeglasses and a method for eyeglasses.

### BACKGROUND

People tend to have little time for physical exercise and find it difficult to commit to a schedule. They may plan to go to the gym but then something comes up and they skip it week after week. In their daily routines, people also often miss simple opportunities to stay active or exercise because they are not mindful about them. Periodic notifications (e.g., daily notifications) that remind people to do certain tasks are easily ignored when they arrive at the wrong time. For example, a notification to walk 10000 steps in the day is not very helpful when a person is in a meeting.

Hence, there may be a demand for situational recognition of opportunities to perform physical health exercises and an appealing presentation to the user.

### SUMMARY

This demand is met by eyeglasses and a method for eyeglasses in accordance with the independent claims. Advantageous embodiments are defined by the dependent claims.

According to a first aspect, the present disclosure provides eyeglasses. The eyeglasses comprise a display and a camera configured to capture an environment of the eyeglasses. Additionally, the eyeglasses comprise processing circuitry coupled to the camera and the display. The processing circuitry is configured to determine, based on image data of the camera, whether the environment offers a user of the eyeglasses an opportunity to perform a physical health exercise. If it is determined that the environment offers an opportunity to perform a physical health exercise, the processing circuitry is configured to control the display to output one or more graphical elements related to the opportunity to perform a physical health exercise.

According to a second aspect, the present disclosure provides a method for eyeglasses. The method comprises capturing an environment of the eyeglasses by a camera of the eyeglasses. Additionally, the method comprises determining, based on image data of the camera, whether the environment offers a user of the eyeglasses an opportunity to perform a physical health exercise. If it is determined that the environment offers an opportunity to perform a physical health exercise, the method further comprises controlling a display of the eyeglasses to output one or more graphical elements related to the opportunity to perform a physical health exercise.

### BRIEF DESCRIPTION OF THE FIGURES

Some examples of apparatuses and/or methods will be described in the following by way of example only, and with reference to the accompanying figures, in which

FIG. 1 illustrates an example of eyeglasses;

FIG. 2 illustrates an example of an augmented environment; and

FIG. 3 illustrates a flowchart of an example of a method for eyeglasses.

### DETAILED DESCRIPTION

Some examples are now described in more detail with reference to the enclosed figures. However, other possible examples are not limited to the features of these embodiments described in detail. Other examples may include modifications of the features as well as equivalents and alternatives to the features. Furthermore, the terminology used herein to describe certain examples should not be restrictive of further possible examples.

Throughout the description of the figures same or similar reference numerals refer to same or similar elements and/or features, which may be identical or implemented in a modified form while providing the same or a similar function. The thickness of lines, layers and/or areas in the figures may also be exaggerated for clarification.

When two elements A and B are combined using an “or”, this is to be understood as disclosing all possible combinations, i.e. only A, only B as well as A and B, unless expressly defined otherwise in the individual case. As an alternative wording for the same combinations, “at least one of A and B” or “A and/or B” may be used. This applies equivalently to combinations of more than two elements.

If a singular form, such as “a”, “an” and “the” is used and the use of only a single element is not defined as mandatory either explicitly or implicitly, further examples may also use several elements to implement the same function. If a function is described below as implemented using multiple elements, further examples may implement the same function using a single element or a single processing entity. It is further understood that the terms “include”, “including”, “comprise” and/or “comprising”, when used, describe the presence of the specified features, integers, steps, operations, processes, elements, components and/or a group thereof, but do not exclude the presence or addition of one or more other features, integers, steps, operations, processes, elements, components and/or a group thereof.

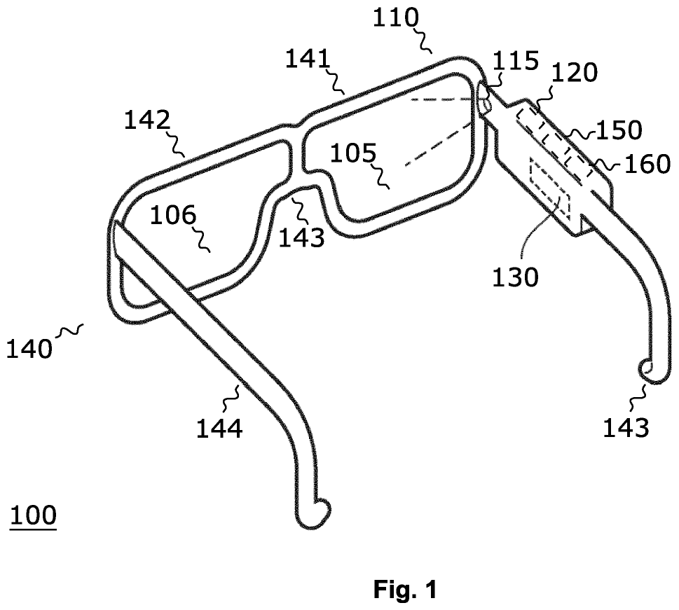

FIG. 1 illustrates exemplary eyeglasses 100. As will be apparent from the following description, the eyeglasses 100 may be understood as smart glasses, i.e., as a head-wearable (head-mountable) computing device.

The eyeglasses 100 comprise a frame 140 adapted for mounting to the head of a user (wearer) of the eyeglasses 100. As illustrated in FIG. 1, the frame 140 comprises two frame portions 141 and 142 for holding lenses 105 and 106, a bridge 143 between the two frame portions 141 and 142 for holding the frame 140 on the user's nose and two hinged arms (temples) 144 and 145 for resting the frame 140 over the user's ears. It is to be noted that the eyeglasses 100 need not comprise two lenses as illustrated in FIG. 1. In other examples, the eyeglasses 100 may comprise only a single lens. The frame 140 may be made of (comprise) various materials such as plastic, polymer, metal or natural materials (e.g., wood).

The eyeglasses 100 further comprise a display 110, a camera 120 and processing circuitry 130. The frame 140 is configured to hold the display 110, the camera 120 and the processing circuitry 130. For example, one of the temples 143 and 144 such as the temple 143 in the example of FIG. 1 may comprise a portion for holding (integrating) at least part of the display 110, the camera 120 and the processing circuitry 130. It is to be noted that the display 110, the camera 120, the processing circuitry 130 and also other elements of the eyeglasses 100 may be arranged in or on the frame as desired or needed. In other words, the display 110, the camera 120, the processing circuitry 130 and also other elements of the eyeglasses 100 may be arranged in or at portions of the frame 140 different from those illustrated in FIG. 1.

The display 110 is an output device for visual presentation of information to the user of the eyeglasses 100. The display 110 may be implemented in various ways. In the example of FIG. 1, the lens 105 of the eyeglasses 100 serves as display 110 together with an optical projector 115. The optical projector 115 is configured to project one or more graphical elements onto the lens 105 such that the user can visually perceive the information represented by the one or more graphical elements. However, it is to be noted that the present disclosure is not limited thereto. In other examples, the display 110 may be formed by an optical source configured to output light representing the one or more graphical elements, and one or more lenses of the eyeglasses 100 (such as the lens 105) including one or more waveguides for guiding the light and controllably emitting the light to the user such that the user can visually perceive the information represented by the one or more graphical elements. In still other examples, a display separate from the lenses of the eyeglasses 100 may be used. As is apparent from the above and the below description, the proposed technology does not depend on the actual implementation of the display 110.

The camera 120 is configured to capture an environment (surrounding) of the eyeglasses 100. For example, the camera 120 may be configured to generate a still image depicting the environment, or an image stream (video stream) depicting the environment over time. The camera 120 is configured to output image data representing (indicative of, encoded with) one or more captured images of the environment.

The processing circuitry 130 is coupled to the camera 120 and the display 110. The processing circuitry 130 is configured to receive and process the image data of the camera 120. Furthermore, the processing circuitry 130 is configured to control the display 110. For example, the processing circuitry 130 may be a single dedicated processor, a single shared processor, or a plurality of individual processors, some of which or all of which may be shared, a digital signal processor (DSP) hardware, an application specific integrated circuit (ASIC), a neuromorphic processor or a field programmable gate array (FPGA). The processing circuitry 130 may optionally be coupled to, e.g., read only memory (ROM) for storing software, random access memory (RAM) and/or non-volatile memory.

In particular, the processing circuitry 130 is configured to determine, based on the image data of the camera 120, whether the environment offers the user of the eyeglasses 100 an opportunity to perform a physical health exercise. In other words, the processing circuitry 130 is configured to determine whether the environment of the user is favorable for performing a physical health exercise. A physical health exercise is a physical (bodily) activity for enhancing or maintaining a physical fitness or overall health and wellness of a person such as the user. Examples of a physical health exercise may, e.g., be a running exercise, a jumping exercise, a pushup exercise, a weightlifting exercise, or a combination thereof. For example, the processing circuitry 130 may be configured to determine whether the environment is favorable for performing a running and/or weightlifting exercise. Since many different physical health exercises are conceivable, there are also multitudes of criteria for determining whether the environment offers the user an opportunity to perform a physical health exercise. Some examples will be described below.

If it is determined that the environment offers an opportunity to perform a physical health exercise, the processing circuitry 130 is further configured to control the display 110 to output one or more graphical elements related to the opportunity to perform a physical health exercise. The one or more graphical elements may represent various information related to the opportunity to perform a physical health exercise. For example, the one or more graphical elements may indicate that performing a physical health exercise is possible or advised in the current environment. Alternatively or additionally, the one or more graphical elements may indicate the type of physical health exercise the user may perform and optionally further indicate guidance on how to perform the physical health exercise (e.g., a video or any other kind of tutorial). In general, the one or more graphical elements may indicate any type of information that enables the user to perform and/or supports the user in performing the physical health exercise.

The eyeglasses 100 may recognize opportunities to perform physical health exercises and remind the user to do some exercise and stay active when the situation is right. The eyeglasses 100 may avoid situations in which the user is reminded to perform an exercise at the wrong time. Furthermore, the eyeglasses 100 may support the user in performing the physical health exercise.

For determining whether the environment offers an opportunity to perform a physical health exercise, the processing circuitry 130 may be configured to perform image analysis on the image data of the camera 120. Image analysis refers to the process of extracting meaningful information or insights from the image data using computational methods. For example, specific portions of one or more images represented by the image data may be segmented and analyzed. Various analysis steps such as 2D and 3D object recognition, motion detection, etc. may be applied. The image analysis processing allows to detect and identify objects or patterns within the image(s), measure various characteristics of the depicted environment (e.g., color, texture, or brightness), or classify the image(s) based on its content or context. In particular, the image analysis allows extraction of information from the image data of the camera 120 that enables determination whether the user's environment offers an opportunity to perform a physical health exercise. For example, the image analysis allows determination of one or more of a user's current location (e.g., at home, at the bus stop, at the grocery store), a user's current movement type (standing still, sitting, laying, walking, running), a topological structure of the user's environment (e.g., flat environment, environment with elevations-artificial or natural), an accessibility of the user's environment (e.g., degree people or obstacles around the user) and suitable objects for physical health exercises in the user's environment. These pieces of information allow to determine whether the environment offers an opportunity to perform a physical health exercise as will become evident from the following examples.

For example, the processing circuitry 130 may be configured to determine that the environment offers an opportunity to perform a physical health exercise if a change of an angle of view in the image data is less than a predefined threshold during a time interval (e.g., 1, 5 or 10 minutes). The angle of view refers to the angular extent of the environment that the camera 120 captures and records in each image of the image data. If the angle of view does not change significantly over time, the user is likely to look or stare at the same object for a long time. For example, if the user is at work and stares at his/her computer screen without (significantly) moving the head for a longer period of time, the processing circuitry 130 determines from the image data that the environment offers an opportunity to perform a physical health exercise. By outputting the one or more corresponding graphical elements via the display 110, the eyeglasses 100 may make the user move occasionally and allow the user to enhance his/her health and wellbeing. The predefined threshold as well as the time interval may be selected based on a desired degree of activity for the user.

Alternatively or additionally, the processing circuitry 130 may be configured to determine that the environment offers an opportunity to perform a physical health exercise if the processing circuitry 130 determines from the image data that the user is standing still. For example, while the user is waiting a bus stop, the processing circuitry 130 may determine that the environment offers an opportunity to perform a physical health exercise such that the user can use the waiting time at the bus stop for a physical health exercise. The processing circuitry 130 may, e.g., be configured to determine a position of the user over time from the images represented by the image data and determine that the user is standing still if the position changes less than a predefined threshold during a time interval (e.g., 30 or 60 seconds).

Further alternatively or additionally, the processing circuitry 130 may be configured to determine that the environment offers an opportunity to perform a physical health exercise if the processing circuitry determines from the image data that the user is moving to a predefined location. The predefined location may, e.g., be a location such as a park, a grocery store or the post office. The processing circuitry 130 may, e.g., be configured to determine one or more buildings, streets, stores, landmarks, etc. from the images represented by the image data and derive or estimate the destination of the user. For example, if the processing circuitry 130 determines from the image data that the user passes certain buildings, streets, stores, landmarks, etc. over time, the processing circuitry 130 may determine that the user is moving to a predefined location such as a specific grocery store or a specific park. In another example, if the processing circuitry 130 determines that the user is carrying a parcel, the processing circuitry 130 may determine that the user is moving to the post office as an exemplary predefined location. Similarly, if the processing circuitry 130 determines that the user is carrying a shopping bag, the processing circuitry 130 may determine that the user is moving to a predefined location such as a grocery store or a supermarket. The path to the predefined location may be used for performing physical health exercises by the user. For example, the path to the predefined location may be used for a running exercise. By running rather than walking to the predefined location, the user may train his/her physical fitness.

It is to be noted that the processing circuitry 130 may consider further input for determining whether the environment offers an opportunity to perform a physical health exercise. For example, the eyeglasses 100 may comprise one or more further sensors 150 such as a positioning sensor (e.g., for a navigation satellite constellation such as the Global Positioning System (GPS), GLObalnaya NAvigatSionnaya Sputnikovaya Sistema (GLONASS), Galileo and/or BeiDou), an acceleration sensor or an inertial measurement unit. Accordingly, the processing circuitry 130 may be configured to determine whether the environment offers an opportunity to perform a physical health exercise further based on measured sensor data of one or more of these additional sensors. For example, the sensor data of an additional positioning sensor may be used for determining whether the user is standing still or determining whether the user is moving to a predefined location. Similarly, the sensor data of an acceleration sensor or an inertial measurement unit may be used for determining whether the user is standing still. However, it is to be noted that the above listed types of additional sensors are for illustrative purposes only. Other sensors may be used as well. Further, it is to be noted that the additional sensor(s) need not be part of the eyeglasses 100. In some examples, the eyeglasses 100 may comprise circuitry for receiving measured sensor data from one or more sensors external to the eyeglasses 100. In the example of FIG. 1, the eyeglasses 100 comprise additional wireless communication circuitry 160 configured or configurable to wirelessly receive measured sensor data from one or more sensors external to the eyeglasses 100 (e.g., from one or more sensors of a mobile phone of the user). However, it is to be noted that the wireless communication circuitry 160 is optional.

As described above, the one or more graphical elements output by the display 110 may indicate a suggested physical health exercise for the user which includes physical movement of the user. For example, while the user is waiting a bus stop, the processing circuitry 130 may determine that the environment offers an opportunity to perform a physical health exercise and control the display to output one or more graphical elements suggesting the user to perform some squats, perform shadow boxing, jog or run in the vicinity of the bus stop or perform a dance. In another example, while the user is walking to a grocery store which is a predefined location, the processing circuitry 130 may determine that the environment offers an opportunity to perform a physical health exercise and control the display 110 to output one or more graphical elements suggesting the user run to the grocery store rather than walk.

The processing circuitry 130 may further be configured to determine that the environment offers an opportunity to perform a physical health exercise if the processing circuitry 130 determines from the image data that the user is carrying a load. For example, the processing circuitry 130 may perform object recognition processing on the image data to determine whether the user is carrying a load such as a shopping bag, a parcel, a sack etc. The load may be used by the user for performing physical health exercises such as weightlifting exercises. Accordingly, the one or more graphical elements may indicate a suggested physical health exercise for the user which involves the load. For example, the one or more graphical elements may indicate that the user should lift the load for a few times or do a farmer's carry exercise to train his/her arm and/or should muscles. To support the user in performing the suggested physical health exercise, the one or more graphical elements may comprise a video or any other kind of tutorial for performing the suggested physical health exercise.

Alternatively or additionally, the processing circuitry 130 may be configured to determine that the environment offers an opportunity to perform a physical health exercise if the processing circuitry 130 determines from the image data that the environment provides, for the user, two or more alternative paths requiring different levels of physical activity for reaching a target location. The processing circuitry 130 may, e.g., perform object recognition processing on the image data to determine whether alternative paths for reaching a target location are present in the environment. For example, if the image data show that an escalator and a stairway are located in the environment for reaching another floor, the processing circuitry 130 may determine that two alternative paths for reaching the other floor (which is the target location in this example) are available. As using the stairway requires more physical activity of the user than using the escalator, the processing circuitry 130 may determine that two or more alternative paths requiring different levels of physical activity for reaching the other floor are available. Using the stairway rather than the escalator allows the user to train his/her physical fitness. Hence, the processing circuitry 130 may determine that the environment offers an opportunity to perform a physical health exercise. To support the user in using the stairway rather than the escalator, the one or more graphical elements may indicate that the escalator is currently out of order. For example, the one or more of the one or more graphical elements may represent a virtual “out-of-order”-sign in front of the escalator and, hence, augment the environment to support the user in using the stairway rather than the escalator. Speaking more general, the one or more graphical elements may indicate that one or more of the alternative paths requiring the least physical activity are not available to support the user in taking a path requiring more physical activity.

As described in the above example for the virtual “out-of-order”-sign, the one or more graphical elements displayed to user via the display 110 may generally represent one or more virtual objects for augmenting the environment to support the user in performing the physical health exercise. For example, to support the user in a running exercise, one or more of the one or more graphical elements may represent a virtual angry dog chasing the user. This will make the physical health exercise more exciting for the user. Similarly, to support the user in performing a dance exercise, one or more of the one or more graphical elements may represent the dance steps to be performed by the user. In another example, to support the user in performing a punching or jumping exercise, one or more of the one or more graphical elements may represent a red ball hovering over the user. In still another examples, to support the user in performing a punching exercise, one or more of the one or more graphical elements may represent a clown face or a punching bag which the user is supposed to punch. In some examples, the one or more of the one or more graphical elements may represent a friend of the user or a ghost version of the user exercising next to the user to motivate the user. Accordingly, the environment of the user may be transferred into an exciting environment for performing a physical health exercise.

According to examples of the present disclosure, the one or more graphical elements may represent information about an effect of the physical health exercise. For example, one or more of the one or more graphical elements may indicate the number of calories burned/burnable by performing the physical health exercise. Alternatively or additionally, one or more of the one or more graphical elements may indicate which muscles are trained/trainable by performing the physical health exercise. In another example, one or more of the one or more graphical elements may indicate how much time the user saved by running instead of walking or how much the user's carbon footprint is reduced by running instead of using the car.

The eyeglasses 100 not only trigger the physical health exercise but may also end the physical health exercise at the right time. The processing circuitry 130 may, e.g., be configured to determine whether the user should end the physical health exercise. If it is determined that the user should end the physical health exercise, the processing circuitry 130 may be further configured to control the display 110 to output one or more graphical elements indicating that the user should end the physical health exercise. For example, if the processing circuitry 130 determines from the image data that the user is close to the predefined location (e.g., the predefined location is already depicted in a captured image represented by the image data), the processing circuitry 130 may determine to end a physical health exercise such as a running exercise. The one or more graphical elements allow to tell the user to stop running. Accordingly, the user can breathe and go calmly before reaching the predefined location (e.g., a grocery store). In case a virtual object for augmenting the environment (e.g. a virtual angry dog chasing the user) is presented to the user via the one or more graphical elements, the presentation of the virtual object may be ended (e.g., the virtual angry dog may run away from the user). In other examples, the processing circuitry 130 may determine that the user should end the physical health exercise if the user performed the physical health exercise for a predefined period of time. For example, the processing circuitry 130 may start a timer once the user starts with the physical health exercise and determine that the user should end the physical health exercise once the timer exceeds the predefined period of time. The start of the exercise may be determined by the processing circuitry 130 from the image data of the camera 120 by means of image analysis. In other examples, the processing circuitry 130 may determine that the user should end the physical health exercise once the user repeated the physical health exercise a predefined number of times. For example, if the user performs a weightlifting exercise, the processing circuitry 130 may determine that the user should end the physical health exercise once the user lifted the load a predefined number of times. The number of repetitions of the physical health exercise may be determined by the processing circuitry 130 from the image data of the camera 120 by means of image analysis. In still other examples, the processing circuitry 130 may determine that the user should end the physical health exercise if the user ran a predefined distance. The distance ran by the user may be determined by the processing circuitry 130 from the image data of the camera 120 by means of image analysis. Additionally or alternatively, the processing circuitry 130 may analyze measured sensor data of one or more of the additional sensors 150 for determining whether the user should end the physical health exercise. For example, the measured sensor data of a positioning sensor or an acceleration sensor may be used to determine the distance run by the user.

The eyeglasses 100 may further support joint (cooperative) physical exercises involving the user and one or more people in the vicinity of the user.

For example, the wireless communication circuitry 160 may be configured to wirelessly receive first availability data from other eyeglasses in the vicinity of the user. The other eyeglasses may be implemented as described herein for the eyeglasses 100. The first availability data indicate that a wearer of the other eyeglasses is available for performing a joint physical health exercise. A joint physical health exercise is a physical health exercise performed together by two or more people. The first availability data may be understood as a beacon signaling the wearer of the other eyeglasses' availability for performing a joint physical health exercise.

If it is determined that the environment offers an opportunity to perform a physical health exercise, the processing circuitry 130 may be further configured to determine a joint physical health exercise for the user and the wearer of the other eyeglasses. For example, the joint physical health exercise may be virtual fencing, virtual boxing, virtual frisbee, dancing, etc. The joint physical health exercise may, e.g., be selected based on the current location of the user and/or the wearer of the other eyeglasses, a topological structure of the environment of the user and/or the wearer of the other eyeglasses and/or an accessibility of the environment of the user and/or the wearer of the other eyeglasses.

Further, the processing circuitry 130 may be configured to control the display 110 to output one or more graphical elements related to the joint physical health exercise. For example, the one or more graphical elements may indicate that there is an opportunity to perform a joint physical health exercise, indicate which joint physical health exercises are available, indicate guidance on how to perform the joint physical health exercise, represent one or more virtual object for augmenting the environment to support the user in performing the joint physical health exercise, etc.

Additionally, the processing circuitry 130 may be configured to control the wireless communication circuitry 160 to wirelessly transmit exercise data to the other eyeglasses. The exercise data indicate information related to the determined joint physical health exercise. For example, the exercise data may indicate the type of the joint physical health exercise, performance or movement data of the user, general synchronization data to synchronize and coordinate the operation of the eyeglasses 100 and the other eyeglasses with respect to the joint physical health exercise (e.g., synchronization and coordination of output of instructions or virtual objects to the user and the wearer of the other eyeglasses), etc.

Accordingly, the eyeglasses 100 and the other eyeglasses may enable the user and the wearer of the other eyeglasses to perform a joint physical health exercise.

It is to be noted that the user may perform a joint physical health exercise with more than one other person. For example, if three, four or more people with suitable eyeglasses are in the vicinity, the eyeglasses may allow them to perform a joint physical health exercise analogously to what is described above for two people performing a joint physical health exercise.

The wireless communication circuitry 160 may further be configured to wirelessly transmit second availability data to the vicinity of the user. The second availability data indicate that the user is available for performing a joint physical health exercise. Accordingly, eyeglasses of other people in the vicinity may recognize the eyeglasses 100 and initiate a joint physical health exercise involving the user of the eyeglasses 100. The second availability data may be understood as a beacon signaling the user's availability for performing a joint physical health exercise.

The eyeglasses 100 may comprise further elements such as, e.g., a human-machine interface for enabling user inputs of the user (e.g., one or more buttons, sliders and/or touch-sensitive surfaces), memory, a loudspeaker, an audio driver, further sensors, a power management integrated circuit or a smart battery.

The wireless communication circuitry 160 of the eyeglasses 100 may be configured to operate according to one of the 3rd Generation Partnership Project (3GPP)-standardized mobile communication networks or systems. The mobile or wireless communication system may correspond to, for example, a 5th Generation New Radio (5G NR), a Long-Term Evolution (LTE), an LTE-Advanced (LTE-A), High Speed Packet Access (HSPA), a Universal Mobile Telecommunication System (UMTS) or a UMTS Terrestrial Radio Access Network (UTRAN), an evolved-UTRAN (e-UTRAN), a Global System for Mobile communication (GSM), an Enhanced Data rates for GSM Evolution (EDGE) network, or a GSM/EDGE Radio Access Network (GERAN). Alternatively or additionally, the wireless communication circuitry 160 may be configured to operate according to mobile communication networks with different standards, for example, a Worldwide Inter-operability for Microwave Access (WIMAX) network IEEE 802.16 or Wireless Local Area Network (WLAN) IEEE 802.11, a Bluetooth network IEEE 802.15.1, a Near Field Communication (NFC) network, generally an Orthogonal Frequency Division Multiple Access (OFDMA) network, a Time Division Multiple Access (TDMA) network, a Code Division Multiple Access (CDMA) network, a Wideband-CDMA (WCDMA) network, a Frequency Division Multiple Access (FDMA) network, a Spatial Division Multiple Access (SDMA) network, etc.

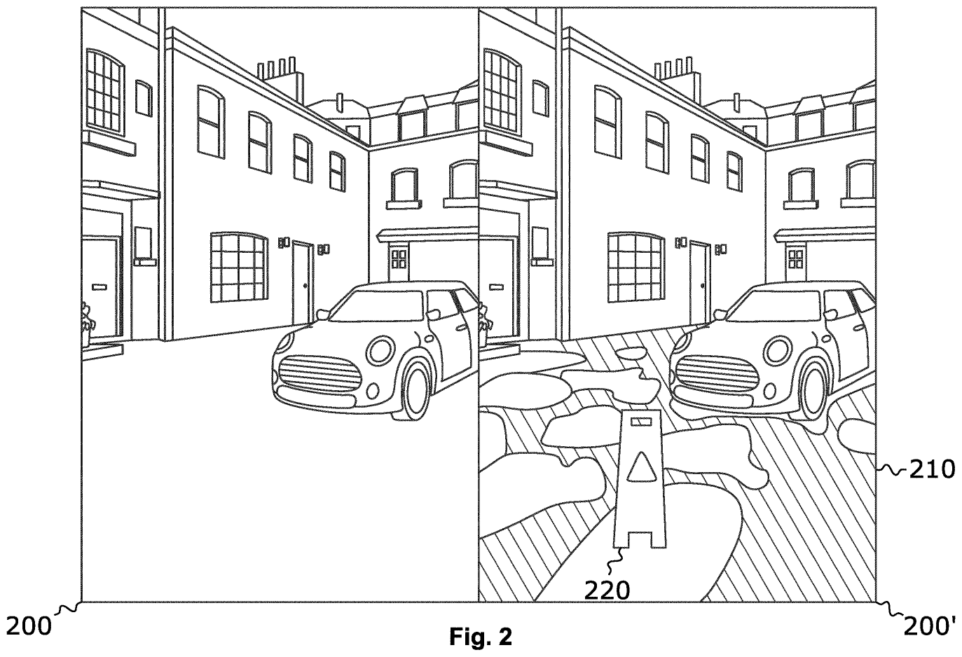

As described above, the processing circuitry 130 may control the display 110 to output one or more graphical elements representing one or more virtual objects for augmenting the environment to support the user in performing the physical health exercise. This is exemplarily illustrated in FIG. 2. The left part of FIG. 2 illustrates the environment 200 of the user as it is captured by the camera 120. The right part of FIG. 2 illustrates the augmented environment 200′ presented to the user by the eyeglasses 100. In the example of FIG. 2, graphical elements 210 representing virtual lava are output by the display 110 such that the user has the impression that the ground in the environment is covered with lava except for a few spots.

The graphical elements 210 representing the virtual lava are used to support the user in performing, e.g., a jumping exercise. Some spots of the ground are not covered by the virtual lava such that the user can jump between these spots when performing the jumping exercise. Additionally, one or more virtual object such as the warning label 220 may be present to the user via the graphical elements to provide contextual information for the physical health exercise.

The augmented environment 200′ is more exciting for the user and, hence, allows to motivate the user to perform the physical health exercise.

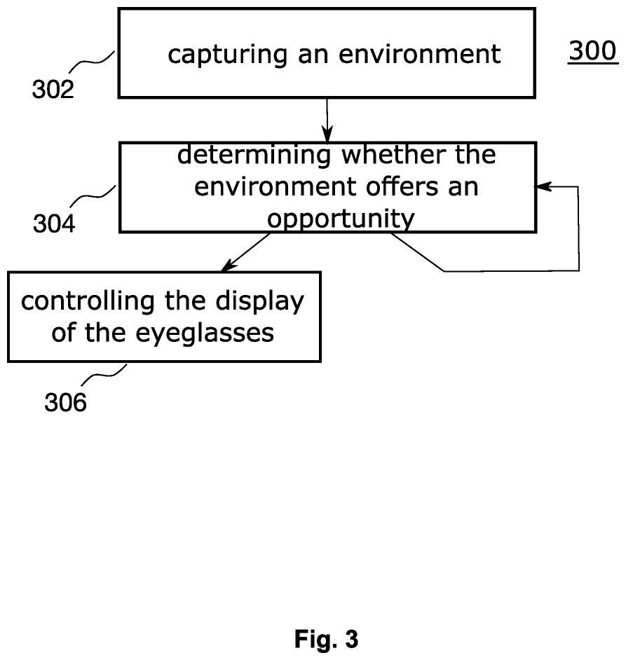

For further highlighting the proposed technology described above, FIG. 3 illustrates a flowchart of a method 300 for eyeglasses such as the eyeglasses 100 described above. The method 300 comprises capturing 302 an environment of the eyeglasses by a camera of the eyeglasses. Additionally, the method 300 comprises determining 304, based on image data of the camera, whether the environment offers a user of the eyeglasses an opportunity to perform a physical health exercise. If it is determined that the environment offers an opportunity to perform a physical health exercise, the method 300 further comprises controlling 306 a display of the eyeglasses to output one or more graphical elements related to the opportunity to perform a physical health exercise.

Analogously to what is described above, the method 300 may allow to recognize opportunities to perform physical health exercises and present them to the user in an appealing manner.

More details and aspects of the method 300 are explained in connection with the proposed technique or one or more examples described above (e.g., FIG. 1 and FIG. 2). The method 300 may comprise one or more additional optional features corresponding to one or more aspects of the proposed technique or one or more examples described above. For example, if it is determined that the environment does not offer an opportunity to perform a physical health exercise, the method 300 may return to determining 304 whether the environment offers the user of the eyeglasses an opportunity to perform a physical health exercise.

The following examples pertain to further embodiments:

(1) Eyeglasses, comprising:
a display;
a camera configured to capture an environment of the eyeglasses; and
processing circuitry coupled to the camera and the display, wherein the processing circuitry is configured to:
   determine, based on image data of the camera, whether the environment offers a user of the eyeglasses an opportunity to perform a physical health exercise; and
   if it is determined that the environment offers an opportunity to perform a physical health exercise, control the display to output one or more graphical elements related to the opportunity to perform a physical health exercise.

(2) The eyeglasses of (1), wherein, for determining whether the environment offers an opportunity to perform a physical health exercise, the processing circuitry is configured to perform image analysis on the image data.

(3) The eyeglasses of (2), wherein the processing circuitry is configured to determine that the environment offers an opportunity to perform a physical health exercise if a change of an angle of view in the image data is less than a predefined threshold during a time interval.

(4) The eyeglasses of (2) or (3), wherein the processing circuitry is configured to determine that the environment offers an opportunity to perform a physical health exercise if the processing circuitry determines from the image data that the user is standing still.

(5) The eyeglasses of (3) or (4), wherein the processing circuitry is configured to determine that the environment offers an opportunity to perform a physical health exercise if the processing circuitry determines from the image data that the user is moving to a predefined location.

(6) The eyeglasses of any one of (2) to (4), wherein the one or more graphical elements indicate a suggested physical health exercise for the user which includes physical movement of the user.

(7) The eyeglasses of any one of (2) to (6), wherein the processing circuitry is configured to determine that the environment offers an opportunity to perform a physical health exercise if the processing circuitry determines from the image data that the user is carrying a load.

(8) The eyeglasses of (7), wherein the one or more graphical elements indicate a suggested physical health exercise for the user which involves the load.

(9) The eyeglasses of any one of (2) to (8), wherein the processing circuitry is configured to determine that the environment offers an opportunity to perform a physical health exercise if the processing circuitry determines from the image data that the environment provides, for the user, two or more alternative paths requiring different levels of physical activity for reaching a target location.

(10) The eyeglasses of (9), wherein the one or more graphical elements indicate that one or more of the alternative paths requiring the least physical activity are not available.

(11) The eyeglasses of any one of (1) to (10), wherein the one or more graphical elements represent one or more virtual objects for augmenting the environment to support the user in performing the physical health exercise.

(12) The eyeglasses of any one of (1) to (11), wherein the one or more graphical elements represent information about an effect of the physical health exercise.

(13) The eyeglasses of any one of (1) to (12), wherein the processing circuitry is further configured to:
determine whether the user should end the physical health exercise; and
if it is determined that the user should end the physical health exercise, control the display to output one or more graphical elements indicating that the user should end the physical health exercise.

(14) The eyeglasses of any one of (1) to (13), further comprising:
wireless communication circuitry configured to wirelessly receive first availability data from other eyeglasses in the vicinity of the user, the first availability data indicating that a wearer of the other eyeglasses is available for performing a joint physical health exercise,
wherein, if it is determined that the environment offers an opportunity to perform a physical health exercise, the processing circuitry is further configured to:
determine a joint physical health exercise for the user and the wearer of the other eyeglasses;
control the display to output one or more graphical elements related to the joint physical health exercise; and
control the wireless communication circuitry to wirelessly transmit exercise data to the other eyeglasses, the exercise data indicating information related to the determined joint physical health exercise.

(15) The eyeglasses of any one of (1) to (14), further comprising:
wireless communication circuitry configured to wirelessly transmit second availability data to the vicinity of the user, the second availability data indicating that the user is available for performing a joint physical health exercise.

(16) The eyeglasses of any one of (1) to (15), further comprising:
a frame configured to hold the display, the camera and the processing circuitry, wherein the frame is adapted for mounting to the user's head.

(17) A method for eyeglasses, the method comprising:
capturing an environment of the eyeglasses by a camera of the eyeglasses;
determining, based on image data of the camera, whether the environment offers a user of the eyeglasses an opportunity to perform a physical health exercise; and
if it is determined that the environment offers an opportunity to perform a physical health exercise, controlling a display of the eyeglasses to output one or more graphical elements related to the opportunity to perform a physical health exercise.

The aspects and features described in relation to a particular one of the previous examples may also be combined with one or more of the further examples to replace an identical or similar feature of that further example or to additionally introduce the features into the further example.

Examples may further be or relate to a (computer) program including a program code to execute one or more of the above methods when the program is executed on a computer, processor or other programmable hardware component. Thus, steps, operations or processes of different ones of the methods described above may also be executed by programmed computers, processors or other programmable hardware components. Examples may also cover program storage devices, such as digital data storage media, which are machine-, processor- or computer-readable and encode and/or contain machine-executable, processor-executable or computer-executable programs and instructions. Program storage devices may include or be digital storage devices, magnetic storage media such as magnetic disks and magnetic tapes, hard disk drives, or optically readable digital data storage media, for example. Other examples may also include computers, processors, control units, (field) programmable logic arrays ((F)PLAs), (field) programmable gate arrays ((F)PGAs), graphics processor units (GPU), application-specific integrated circuits (ASICs), integrated circuits (ICs) or system-on-a-chip (SoCs) systems programmed to execute the steps of the methods described above.

It is further understood that the disclosure of several steps, processes, operations or functions disclosed in the description or claims shall not be construed to imply that these operations are necessarily dependent on the order described, unless explicitly stated in the individual case or necessary for technical reasons. Therefore, the previous description does not limit the execution of several steps or functions to a certain order. Furthermore, in further examples, a single step, function, process or operation may include and/or be broken up into several sub-steps, -functions, -processes or -operations.

If some aspects have been described in relation to a device or system, these aspects should also be understood as a description of the corresponding method. For example, a block, device or functional aspect of the device or system may correspond to a feature, such as a method step, of the corresponding method. Accordingly, aspects described in relation to a method shall also be understood as a description of a corresponding block, a corresponding element, a property or a functional feature of a corresponding device or a corresponding system.

The following claims are hereby incorporated in the detailed description, wherein each claim may stand on its own as a separate example. It should also be noted that although in the claims a dependent claim refers to a particular combination with one or more other claims, other examples may also include a combination of the dependent claim with the subject matter of any other dependent or independent claim. Such combinations are hereby explicitly proposed, unless it is stated in the individual case that a particular combination is not intended. Furthermore, features of a claim should also be included for any other independent claim, even if that claim is not directly defined as dependent on that other independent claim.

What is claimed is:

1. Eyeglasses, comprising:
a display;
a camera configured to capture an environment of the eyeglasses; and
processing circuitry coupled to the camera and the display, wherein the processing circuitry is configured to:
determine, based on image data of the camera, whether the environment offers a user of the eyeglasses an opportunity to perform a physical health exercise; and
if it is determined that the environment offers an opportunity to perform a physical health exercise, control the display to output one or more graphical elements related to the opportunity to perform a physical health exercise.

2. The eyeglasses of claim 1, wherein, for determining whether the environment offers an opportunity to perform a physical health exercise, the processing circuitry is configured to perform image analysis on the image data.

3. The eyeglasses of claim 2, wherein the processing circuitry is configured to determine that the environment offers an opportunity to perform a physical health exercise if a change of an angle of view in the image data is less than a predefined threshold during a time interval.

4. The eyeglasses of claim 2, wherein the processing circuitry is configured to determine that the environment offers an opportunity to perform a physical health exercise if the processing circuitry determines from the image data that the user is standing still.

5. The eyeglasses of claim 3, wherein the processing circuitry is configured to determine that the environment offers an opportunity to perform a physical health exercise if the processing circuitry determines from the image data that the user is moving to a predefined location.

6. The eyeglasses of claim 2, wherein the one or more graphical elements indicate a suggested physical health exercise for the user which includes physical movement of the user.

7. The eyeglasses of claim 2, wherein the processing circuitry is configured to determine that the environment offers an opportunity to perform a physical health exercise if the processing circuitry determines from the image data that the user is carrying a load.

8. The eyeglasses of claim 7, wherein the one or more graphical elements indicate a suggested physical health exercise for the user which involves the load.

9. The eyeglasses of claim 2, wherein the processing circuitry is configured to determine that the environment offers an opportunity to perform a physical health exercise if the processing circuitry determines from the image data that the environment provides, for the user, two or more alternative paths requiring different levels of physical activity for reaching a target location.

10. The eyeglasses of claim 9, wherein the one or more graphical elements indicate that one or more of the alternative paths requiring the least physical activity are not available.

11. The eyeglasses of claim 1, wherein the one or more graphical elements represent one or more virtual objects for augmenting the environment to support the user in performing the physical health exercise.

12. The eyeglasses of claim 1, wherein the one or more graphical elements represent information about an effect of the physical health exercise.

13. The eyeglasses of claim 1, wherein the processing circuitry is further configured to:
determine whether the user should end the physical health exercise; and
if it is determined that the user should end the physical health exercise, control the display to output one or more graphical elements indicating that the user should end the physical health exercise.

14. The eyeglasses of claim 1, further comprising:
wireless communication circuitry configured to wirelessly receive first availability data from other eyeglasses in the vicinity of the user, the first availability data indicating that a wearer of the other eyeglasses is available for performing a joint physical health exercise,
wherein, if it is determined that the environment offers an opportunity to perform a physical health exercise, the processing circuitry is further configured to:
determine a joint physical health exercise for the user and the wearer of the other eyeglasses;
control the display to output one or more graphical elements related to the joint physical health exercise; and
control the wireless communication circuitry to wirelessly transmit exercise data to the other eyeglasses, the exercise data indicating information related to the determined joint physical health exercise.

15. The eyeglasses of claim 1, further comprising:
wireless communication circuitry configured to wirelessly transmit second availability data to the vicinity of the user, the second availability data indicating that the user is available for performing a joint physical health exercise.

16. The eyeglasses of claim 1, further comprising:
a frame configured to hold the display, the camera and the processing circuitry, wherein the frame is adapted for mounting to the user's head.

17. A method for eyeglasses, the method comprising:
capturing an environment of the eyeglasses by a camera of the eyeglasses;
determining, based on image data of the camera, whether the environment offers a user of the eyeglasses an opportunity to perform a physical health exercise; and
if it is determined that the environment offers an opportunity to perform a physical health exercise, controlling a display of the eyeglasses to output one or more graphical elements related to the opportunity to perform a physical health exercise.
